(19) United States
(12) Patent Application Publication
(10) Pub. No.: US 2009/0313726 A1
(43) Pub. Date: Dec. 17, 2009

(54) ABIOTIC STRESS TOLERANT GENE FROM AVICENNIA MARINA ENCODING A PROTEIN

(75) Inventors: Ajay Parida, Chennai (IN); Gayatri Venkatraman, Chennai (IN)

Correspondence Address: OBLON, SPIVAK, MCCLELLAND MAIER & NEUSTADT, L.L.P., 1940 DUKE STREET, ALEXANDRIA, VA, 22314, US

(73) Assignee: M.S. SWAMINATHAN RESEARCH FOUNDATION, Taramani (IN)

(21) Appl. No.: 12/066,314

(22) PCT Filed: Sep. 7, 2006
(86) PCT No.: PCT/IN06/00339

§ 371 Date: Sep. 8, 2008

(30) Foreign Application Priority Data
Sep. 9, 2005 (IN) 1261/CHE/2005

(51) Int. Cl. A01H 5/10; C12N 15/11; C07K 14/00; C12N 15/63; C12N 15/70; C12N 15/74; C12N 15/82; C12N 5/10; A01H 5/00

(52) U.S. Cl. 800/294; 536/23.1; 530/324; 435/320.1; 435/252.2; 435/252.33; 435/419; 800/298; 800/278; 435/469

(57) ABSTRACT

The present invention relates to an abiotic stress tolerant transgenic plant comprising an Am244 DNA from Avicennia marina. The invention also relates to isolation and characterization of a cDNA corresponding to abiotic stress tolerant gene (Am244 DNA) from Avicennia marina. It further relates to a method for producing abiotic salt-tolerant transgenic plants, plant cells and plant tissues capable of over expressing the Am244 DNA thereby conferring abiotic stress tolerance such as salt, drought and dehydration to otherwise abiotic stress sensitive plant species.

### FIELD OF INVENTION

The present invention relates to transgenic plants exhibiting enhanced tolerance to abiotic stresses such as drought, salt and dehydration. In particular, the transgenic plant comprises an abiotic stress tolerant Am244 DNA derived from Avicennia marina.

### BACKGROUND

Environmental factors such as drought, extreme temperatures, high or fluctuating salinity can affect plant growth and performance and in the case of agronomically important plants this may translate to reduce yield. Increasing soil salinization in irrigated areas has necessitated the identification of crop traits or genes, which confer resistance to salinity, either by conventional breeding or through molecular biology techniques (Munns et al. 2002; Cushman and Bohnert 2000). Hyperosmotic stress, such as that caused by exposure of cells to high concentrations of NaCl causes imbalance of cellular ions, change in turgor pressure and cell volume and alters the activity and stability of macromolecules. Although the basic cellular responses appear to be conserved among all plants, plant species employ a variety of mechanisms to cope with osmotic stress. While extensive work on salinity tolerance in Arabidopsis and Mesembryanthemum has led to the identification of candidate salinity sensitive determinants, these plants are not true halophytes (Zhu 2002; Chauhan et al. 2000).

Mangroves are facultative halophytes and exclude most of the salt in seawater. In addition, some species such as A. marina actively secrete salt. Avicennia is a monotypic pantropical mangrove genus with eight species of which A. marina is widely distributed both latitudinally and longitudinally. The high salt tolerance of A. marina is a consequence of water use efficiency which balances the relation between carbon gains, water loss and ion uptake with the transpiration stream on a low but constant level. A. marina grows in coastal regions where the salt concentration can be as high as 9% (Rao 1987). Regulation of inorganic ions occurs partially by exclusion at the roots and also by excretion via salt glands, the excretion rate for sodium and chloride ions being 0.4 and 0.046 μmol m-2 s-1 (Shimony et al. 1973; Boon and Allaway 1982). It is thus an ideal candidate plant for identifying genes conferring salt and drought tolerance.

### SUMMARY OF INVENTION

The present invention relates to an abiotic stress tolerant transgenic plant comprising Am244 DNA from Avicennia marina. In particular, the invention is directed to transgenic plants exhibiting enhanced tolerance to drought, salt and dehydration. The invention also relates to isolation and characterization of cDNA corresponding to abiotic stress tolerant gene (Am244 gene) derived from Avicennia marina. Further the invention also provides a method for producing abiotic stress-tolerant transgenic plants.

One aspect of the invention relates to an isolated nucleic acid molecule for enhanced tolerance to abiotic stress in a plant having a nucleotide sequence with at least 90% homology to the nucleotide sequence set forth in SEQ ID NO: 1 or SEQ ID NO: 2, wherein said sequence codes for a polypeptide having amino acid sequence as shown in SEQ ID NO: 3.

Another aspect of the invention relates to an isolated nucleic acid molecule for enhanced tolerance to abiotic stress in a plant, wherein said nucleic acid molecule comprises a nucleotide sequence as shown in SEQ ID NO: 1, or SEQ ID NO: 2.

Another aspect of the invention is a polypeptide having an amino acid sequence as shown in SEQ ID NO: 3, wherein said polypeptide is encoded by the nucleic acid molecule having polynucleotide sequence as shown in SEQ ID NO: 1 or SEQ ID NO: 2.

Yet another aspect of the invention provides an expression cassette for enhanced tolerance to abiotic stress in plant, wherein said expression cassette comprises the nucleic acid molecule having polynucleotide sequence as shown in SEQ ID NO: 1 or SEQ ID NO: 2 operably linked to a plant expressible regulatory sequence.

Further the invention also provides a recombinant vector comprising the DNA construct comprising the expression cassette, wherein said expression cassette comprises the nucleic acid molecule having polynucleotide sequence as shown in SEQ ID NO: 1 or SEQ ID NO: 2 operably linked to a plant expressible regulatory sequence.

Yet another aspect of the invention provides a recombinant host cell comprising the recombinant vector wherein the recombinant vector comprises the DNA construct comprising the expression cassette, said expression cassette comprises the nucleic acid molecule having polynucleotide sequence as shown in SEQ ID NO: 1 or SEQ ID NO: 2 operably linked to a plant expressible regulatory sequence.

Yet another aspect of the invention relates to an abiotic stress tolerant transgenic plant or plant cell or plant tissue comprising a polynucleotide sequence as shown in SEQ ID NO: 1 or SEQ ID NO: 2, wherein the expression of said nucleic acid molecule results in the enhanced tolerance to abiotic stress in said plant, plant cell and plant tissue thereof.

Further aspect of the invention is directed to a method of producing an abiotic stress tolerant transgenic plant, said method comprising introducing nucleic acid molecule having polynucleotide sequence as shown in SEQ ID NO: 1 or SEQ ID NO: 2 in plant genome by using transformation method, thereby producing abiotic stress tolerant transgenic plant.

### BRIEF DESCRIPTION OF ACCOMPANYING DRAWINGS

FIG. 1a: Expression pattern of the Am244 transcript in leaves and roots of A. marina seedlings under conditions of salinity stress (500 mM NaCl/top panel) and ABA treatment (100 μM/bottom panel) at different time intervals.

FIG. 1b: Expression pattern of the Am244 transcript in leaves and roots of A, marina seedlings under conditions of NaCl (500 mM), KCl (500 mM) and mannitol (800 mM) treatment at different time intervals.

FIG. 2: T-DNA segment of the plasmid pGFP-Ala-Am244-C1.

FIG. 3: T-DNA segment of the pMyc-Am244-C1.

FIG. 4: Guard cells of pGFP-Ala-Am244-C1 transformed Nicotiana tabacum var. Wisconsin 38 tobacco show localization of the green fluorescence at the plasma membrane and close to the cell wall.

### DESCRIPTION OF THE INVENTION

The present invention relates to an abiotic stress tolerant transgenic plants comprising Am244 DNA from Avicennia marina. In particular, the invention is directed to a transgenic plant exhibiting enhanced tolerance to drought, salt or dehydration. The invention also relates to isolation and characterization of a cDNA corresponding to abiotic stress tolerant gene (Am244 DNA) derived from Avicennia marina. Further the invention is also directed to a method for producing the abiotic stress-tolerant transgenic plants.

One embodiment of the present invention relates to an isolated nucleic acid molecule for enhanced tolerance to abiotic stress in plant having a nucleotide sequence with at least 90% homology to the nucleotide sequence set forth in SEQ ID NO: 1 or SEQ ID NO: 2, wherein said sequence codes for a polypeptide having amino acid sequence as shown in SEQ ID NO: 3.

Yet another embodiment of the invention relates to the isolated nucleic acid molecule having nucleotide sequence as shown in SEQ ID NO: 1.

Yet another embodiment of the invention relates to the isolated nucleic acid molecule having nucleotide sequence as shown in SEQ ID NO: 2.

Still another embodiment of the invention is directed to an isolated nucleic acid molecule encoding a polypeptide comprising an amino acid sequence as shown in SEQ ID NO:3.

Still yet another embodiment of the invention relates to a polypeptide having an amino acid sequence as shown in SEQ ID NO: 3, wherein said polypeptide is encoded by the nucleic acid of the present invention.

In another embodiment the invention provides the isolated nucleic acid molecule having nucleotide sequence as shown in SEQ ID NO: 1 and SEQ ID NO: 2 for enhanced tolerance to abiotic stress such as drought stress, salt stress and dehydration stress in plants.

In yet another embodiment the invention relates to an expression cassette for conferring enhanced tolerance to abiotic stress in a plant, wherein said expression cassette comprises the aforementioned nucleic acid molecule operably linked to a plant expressible regulatory sequence.

Further embodiment provides the regulatory sequence such as CaMV 35S, NOS, OCS, AdhI, AdhII and Ubi-1.

Additional embodiment of the invention relates to a DNA construct comprising the expression cassette having nucleic acid molecule as set forth in SEQ ID NO: 1 or SEQ ID NO: 2.

Aforementioned DNA construct further comprising another expression cassette comprising a selectable marker gene operably linked to the regulatory sequence.

The selectable marker gene such as nptII, hptII, pat and bar can be used for the selection of the transformed plant, plant cell and plant tissues thereof.

Yet another embodiment of the invention provides the DNA construct further comprising another expression cassette comprising a scorable marker gene selected from a group consisting of GUS, GFP, LUC and CAT operably linked to the regulatory sequence.

Yet another embodiment of the invention discloses a recombinant vector comprising the aforementioned DNA constructs wherein the constructs comprises the nucleic acid molecule having polynucleotide sequence as shown in SEQ ID NO: 1 or SEQ ID NO: 2.

Further embodiment of the invention provides the recombinant plant transformation vector comprising the nucleic acid molecule having polynucleotide sequence as shown in SEQ ID NO: 1 or SEQ ID NO: 2.

Still another embodiment of the invention relates to a recombinant host cell comprising the recombinant vector comprising the nucleic acid molecule having polynucleotide sequence as shown in SEQ ID NO: 1 or SEQ ID NO: 2, wherein the host cell can be prokaryotic or eukaryotic cell such as E. coli or Agrobacterium or plant cell.

Various strains of E. coli known in the art such as JM101, DH5α, BL21, HB101, and XL1-Blue can be used for the production of recombinant E. coli cell comprising the nucleic acid molecule having polynucleotide sequence as shown in SEQ ID NO: 1 or SEQ ID NO: 2.

Yet additional embodiment of the invention provides recombinant Agrobacterium cells comprising the nucleic acid molecule having polynucleotide sequence as shown in SEQ ID NO: 1 or SEQ ID NO: 2.

Different Agrobacterium strains provided in the art for example LBA4404, EHA101, EHA105, GV3101 and A281 may be used for the production of the recombinant Agrobacterium.

Additional embodiment of the invention provides a plant cell comprising the nucleic acid molecule having polynucleotide sequence as shown in SEQ ID NO: 1 or SEQ ID NO: 2.

In one preferred embodiment the invention provides an abiotic stress tolerant transgenic plant or plant cell or plant tissue comprising the nucleic acid molecule of the present invention, wherein the expression of the said nucleic acid molecule results in enhanced tolerance to abiotic stress in said plant, plant cell and plant tissue. Further it also provides the progeny derived from the transgenic plants and seeds produced from them.

Yet another preferred embodiment of the invention is directed to a method of producing an abiotic stress tolerant transgenic plant, said method comprising introducing nucleic acid molecule as shown in SEQ ID NO: 1 or SEQ ID NO: 2 in plant genome by using transformation method, thereby producing abiotic stress tolerant transgenic plant.

Still another preferred embodiment of the invention relates to the transformation methods used to develop abiotic stress tolerant transgenic plants.

Plant transformation can be carried out by several methods already known in the art such as Agrobacterium mediated transformation, particle bombardment, vacuum-infiltration, in planta transformation and chemical methods.

Further embodiment of the invention is directed to an Agrobacterium mediated transformation method for producing abiotic stress tolerant transgenic plant, said method comprising:
a) obtaining suitable explants from a plant;
b) constructing the recombinant vector as described in the instant invention;
c) mobilizing said vector in an Agrobacterium cell to produce a recombinant Agrobacterium cell;
d) co-cultivating said explants with said recombinant Agrobacterium cell to produce transformed plant cells,
e) culturing said transformed plant cells to produce abiotic stress-tolerant transgenic plant.

Plants suitable for transformation with the vectors of the invention can be a monocotyledonous and dicotyledonous plant. The monocotyledonous plant is selected from a group consisting of rice, maize, wheat, barley and sorghum. Further the monocotyledonous plant is a rice plant. The dicotyledonous plant is selected from a group consisting of tobacco, tomato, pea, soybean, Brassica, okra, chickpea and pigeon pea. The dicotyledonous plant is a tobacco plant.

A broad range of other monocotyledonous or a dicotyledonous plant including cereal crops, pulse crops, vegetables, and other crops can also be used.

Examples of the moonocotyledonous plant include wheat, rice, barley, maize, oats, millets, sorghum, sugarcane and rye.

Examples of dicotyledonous plant includes pea, chickpea, tobacco, pigeonpea, Arabidopsis, soybean, brinjal, tomato, cucumber, brassicas, cauliflower, cabbage, cotton.

Still another preferred embodiment of the invention relates to the explants used for transformation. Further they are selected from a group consisting of cotyledons, hypocotyls, leaves, anthers, callus, cotyledonary nodes, stems and roots.

Abiotic stress tolerant gene designated as Amb 244 gene derived from A. marina, belongs to the uncharacterized upf0057 family of putative plasma membrane proteins and is found to be strongly upregulated in the present study in response to abiotic stresses. As with Am244, homologous genes identified in other plant species have also been associated with abiotic stress response and this has also been observed for Saccharomyces. In the salt stress tolerant Lophopyrum, ESI3 was upregulated within 2 hours of treatment with 250 mM NaCl and also by treatment with KCl, ABA and osmotic shock (Gulick et al, 1994). The Phytophthora Ric1 gene has been shown to be induced by extremes of pH as well as NaCl treatment (van West et al, 1999). Saccharomyces PMP3 transcript is rapidly and strongly upregulated (17 fold) within 10 minutes of treatment growth in 1M NaCl (Yale and Bohnert, 2001). Further, deletion of the yeast homolog, PMP3 causes salt sensitivity and membrane hyperpolarization and expression of Arabidopsis RCI2A cDNA can complement the pmp3 deletion mutant, indicating that the plant and yeast proteins have similar functions during high salt stress.

A preferred embodiment of the present invention relates to plant growth conditions and RNA and DNA isolation. Seeds of A. marina collected from their natural mangrove habitat Pichavaram, Tamil Nadu, India. Seeds were grown in sand-filled trays in the green house at 37° C. and 12 μl light/dark photoperiod (illuminated from 06:00 to 18:00) in near-submergence conditions and watered daily.

Leaf tissue was harvested and total RNA was isolated according to the method given by Chomczynski & Sacchi, 987. RNA isolation can also be carried out by other methods known in art. Total mRNA can also be extracted using such known protocols optimized for isolation of plant RNA using TRIZOL method. The RNA isolation from A. marina seedling can also be carried out using commercially available plant RNA isolation kits. Details of growth conditions and RNA isolation are given in Example 1

Yet another embodiment of the present invention is directed to a cDNA library construction. The method for synthesis of cDNA and cloning in suitable vectors are well known in art. Several kits are available for cDNA synthesis from (A+) enriched RNA and well known to the person skilled in the art. Kits for cloning cDNA inserts both directionally and randomly are also well known and can be employed. Many kinds of commercially available vectors can alternatively be used for library preparation such as λgt10 and λgt11. The ligated cDNA library was transformed into E. coli DH5α. For further details see Example 2. A library of approximately 10⁵ recombinants was obtained (Parani M, 1999). Plasmid DNA from several randomly selected clones was extracted by alkaline lysis (Feliciello and Chinali 1993). The DNA sequence of the selected clones was determined by using conventional methods of sequencing to generate expressed sequenced tags (ESTs). 10-12 ESTs were randomly selected and analysed for expression (Northern) under salinity stress conditions (0.5M NaCl) in Avicennia marina. One of these genes was found to be up-regulated in both leaves and roots of Avicennia marina a under salinity stress. The clone was designated as Am244 and was selected for further analysis. For details see Example 2.

The polynucleotide sequence of cDNA of Am244 gene is shown in SEQ ID NO: 1. The cDNA of Am244 gene is 600 bp in length and encodes a protein consisting of 57 amino acids (SEQ ID NO: 3). The nucleotide sequence ORE of Am244 cDNA is given in SEQ ID NO: 2.

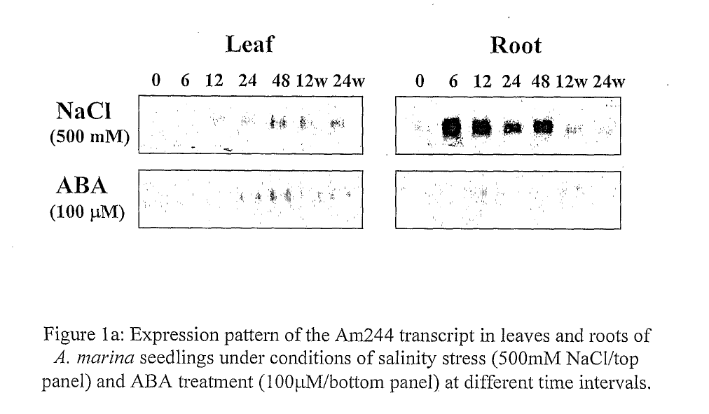

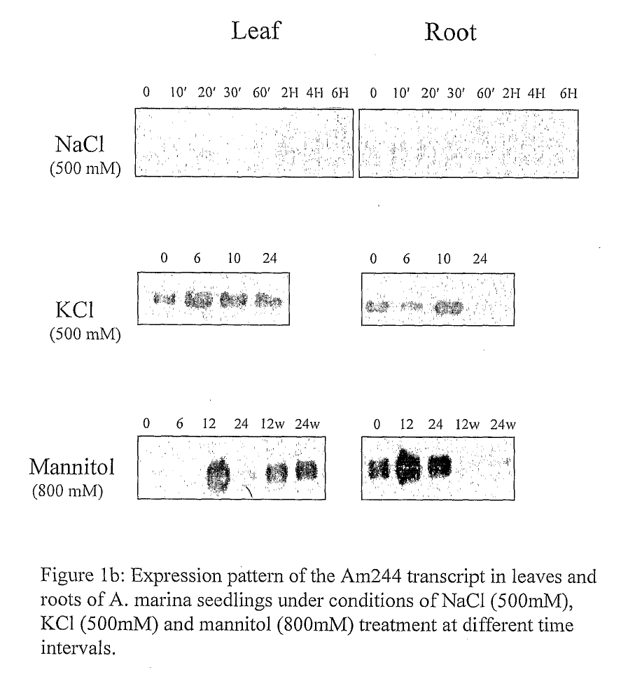

Yet another embodiment relates to the expression analysis of Am244 gene in response to diverse abiotic stresses. Regulation of Am244 gene was analyzed by studying the effect of various abiotic stresses such as NaCl, KCl, ABA and Mannitol. Total RNA was isolated from the plants subjected to various stress condition according to the method given by Chomczynski and Sacchi, 1987 and northern analysis was carried out. Details are provided in Example 3. Am244 was identified to be upregulated for salt, ABA and drought stress. See FIG. 1a and 1b.

Additional embodiment provides the BLAST analysis of Am244 protein. The Am244 protein sequence compared with the protein sequences available in various databases for searching the homology with other related protein sequence which show up-regulation in response to abiotic stress conditions. Details are given in Example 4.

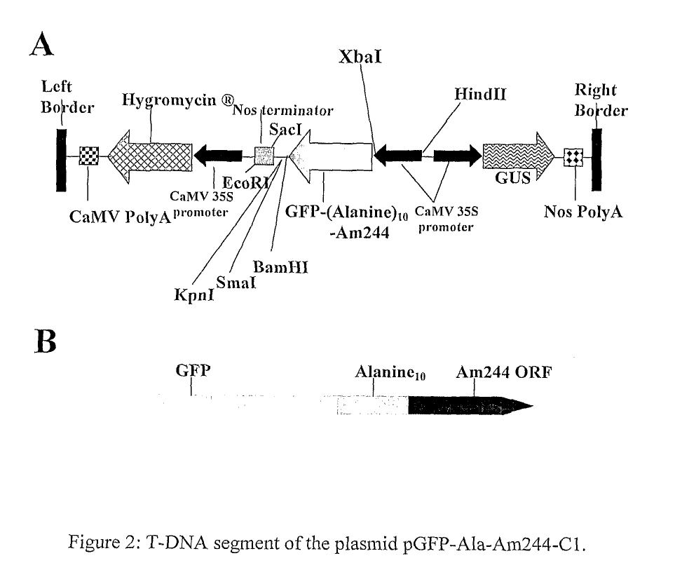

Yet another embodiment of the present invention is directed towards GFP fusion with Am244 cDNA (GFP-Alanine₁₀-Am244) and construction of plant transformation vector comprising a fragment comprising of GFP (Green Fluorescent protein) with a flexible (Alanine)₁₀ linker to the N-terminus of the Am244 ORF (SEQ ID NO: 2). This was accomplished by Splice Overlap Extension (SOEing). (See FIG. 2). Such expression or recombinant vectors may be constructed by methods known in the art.

Various recombinant vectors comprising GFP gene fused to Am244 ORE having nucleotide sequence as shown in SEQ ID NO: 1 or SEQ ID NO: 2 operably linked to the regulatory sequences such as CaMV 35S, NOS, OCS, AdhI, AdhII and Ubi-1. Details are provided in Example 5.

Still yet another embodiment of the invention provides epitope tagging of the Am244 gene. The Am244 ORE codes for a small protein of 57 amino acids of which a substantial part is buried in the plasma membrane as two transmembrane domains. Raising antibodies against membrane spanning proteins is not easy. This can be circumvented by epitope tagging of the Am244 ORF.

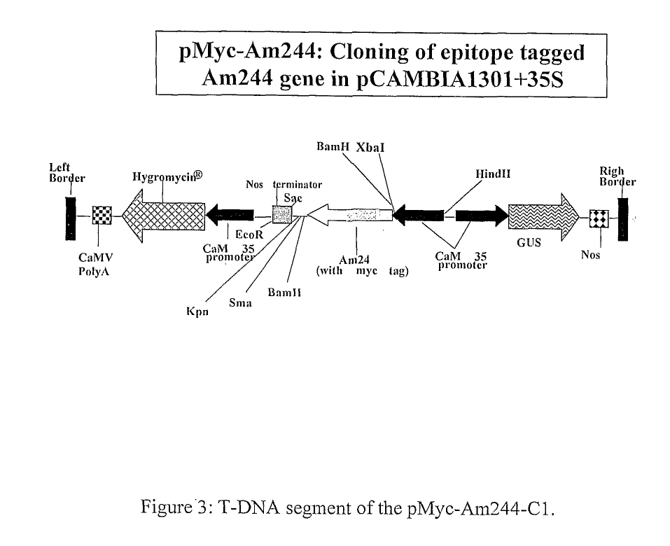

Epitope tagging is a versatile tool used to study proteins, wherein a well characterized peptide tag is fused in-frame with the protein open reading frame (ORE) using recombinant DNA techniques. Epitope tagging can be used to characterize proteins (especially specific members of multigene families), determine subcellular localization, establish topology of membrane proteins, identify interacting partners and track movement within the cell. Epitope-specific commercial antibodies (usually monoclonal) can be then used to address questions about protein localization and function. The c-myc epitope is a well characterized one against which numerous commercially generated monoclonal antibodies are available. The 5′ and 3′ UTRs of the Am244 cDNA were not disturbed and by insertion mutagenesis using partially complementary primers a c-myc epitope was introduced at the N-terminus of the Am244 ORF. Detailed procedure is given in Example 6. (See FIG. 3).

It involves mobilization of various recombinant plant transformation vectors as described above and in the given examples into Agrobacterium tumefaciens strain LBA4404 by the freeze-thaw method. Various other strains of Agrobacterium known in the art such as EHA110, EHA105, A281 may be used for the transformation. For details see Example 5. Yet another embodiment of the present invention is directed to a method for producing abiotic stress tolerant transgenic tobacco plant (Nicotiana tabacum) cv. Petit Havana expressing Am244 DNA from A. marina (See Example 5 and 7 for details).

Other methods known to persons skilled in the art can also be employed for Agrobacterium transformation. Apart from freeze thaw method, one may mobilize the vectors into the Agrobacterium strain also by electroporation or tri-parental mating. All these techniques are well known in the art.

Similarly, transgenic rice plant expressing the Am244 gene from A. marina was produced by using the recombinant vectors disclosed in the invention by the transformation method known in the art. Details are given in Example 8.

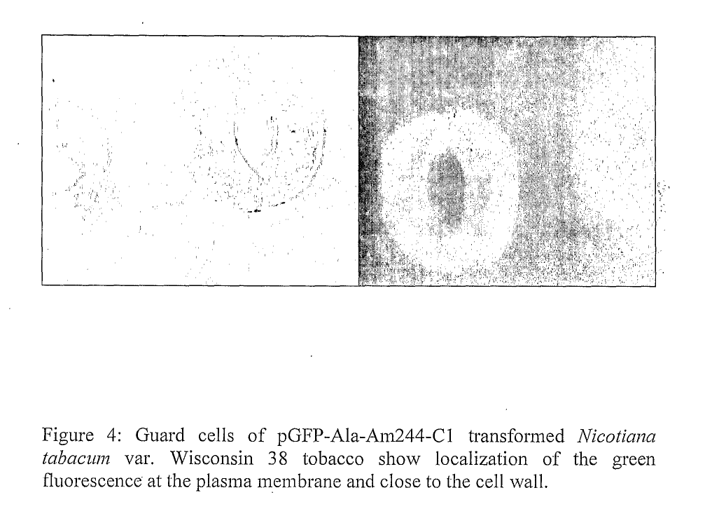

Microscopy and Imaging of the leaf tissue of the transgenic tobacco and rice plant was carried out for the localization of GFP protein in the transgenic plant tissue. Details are given in Example 9. (See FIG. 4).

One embodiment of the invention provides the screening of presence Am244 DNA in transgenic tobacco and rice by PCR using gene specific primers. Other methods well known to persons skilled in the art can also be employed.

Another embodiment of the invention relates to confirming the integration of Am244 DNA in single copy in transgenic tobacco and rice by southern hybridization method (Sambrook et al. 2001). For details see Example 7.

Further embodiment of the invention provides the expression analysis of the transgenic tobacco and rice plants subjected to various abiotic stress treatments. For details refer Example 10.

### EXAMPLES

The examples given are merely illustrative of the uses, processes and products claimed in this invention, and the practice of the invention itself is not restricted to or by the examples described.

### Example 1

### Plant Growth Conditions

Seeds were grown in sand-filled trays in the green house at 37° C. and 12 h light/dark photoperiod (illuminated from 6 hrs to 18 hrs) in near-submergence conditions and watered daily. One-month-old A. marina seedlings (four-leaf stage) were acclimatized for 72 hours in 0.5× Murashige & Skoog (MS) medium (no pH adjustment). Subsequently the plants were transferred into 0.5×MS medium supplemented with 0.5M NaCl for 48 hours.

### RNA Isolation

Leaf and root tissue from plants grown under conditions as mentioned above was harvested and total RNA was isolated according to the method given by Chomczynski and Sacchi, 1987. Leaf and root tissue was harvested from pooled plants and five grams of tissue was macerated in liquid nitrogen and suspended in 18 ml of RNA extraction buffer. To the slurry, 1.8 ml of 2 M sodium acetate (pH 4.0), 18 ml of water saturated phenol and 3.6 ml of 49:1 chloroform:isoamyl alcohol were sequentially added and mixed by inversion. The contents were mixed and cooled on ice for 15 minutes. Finally, the suspension was centrifuged at 10,000×g for 10 minutes at 4° C. After centrifugation, the aqueous phase was transferred to a fresh tube and mixed with equal volume of ice-cold isopropantol and incubated at −20° C. for overniglht. The samples were centrifuged at 10,000×g for 20 minutes at 4° C. and the pellet was dissolved in 5 ml of RNA extraction buffer. The RNA was again re-precipitated with equal volume of ice-cold isopropanol. The pellet was washed in 70% ethanol and finally dissolved in formamide. Purity of the RNA preparation was checked spectrophotometrically by measuring A260/A280 ratio as well as checked for integrity on a formaldehyde-MOPS gel. An A260/A280 value between 1.8 and 2.0 suggested that the RNA was intact and pure. Finally, the total RNA in the samples was estimated by measuring A260. Poly (A⁺) mRNA was isolated by affinity chromatography on oligo (dT)-cellulose as described by Sambrook et al. (1989).

### Example 2

### cDNA Library Construction

cDNA prepared from poly (A+) mRNA using Oligo-dT columns was size fractionated over SizeSep-400 spun column and directionally cloned in the Sal I (5′)/Not I (3′) sites of pSPORT1. The cDNA library was constructed using SuperScript II Reverse Transcriptase and primer-adapters for SalI and NotI enzyme sites enabling cDNA inserts to be directionally cloned in plasmid vectors well known in art. The vector pSPORT1 was utilized for cloning cDNA fragments. The common methods for cDNA synthesis involve using poly (A⁺) RNA as a template for reverse transcription employing an oligo (dT) primer and a reverse transcriptase enzyme to synthesize first strand cDNA. These methods for synthesis of cDNA and cloning in suitable vectors are well known in art. The ligated cDNA library was transformed into E. coli DH5-α strain. E. coli transformation was carried out by the method well known in the art. A library of approximately 10⁵ recombinants was obtained (Parani M, 1999). Plasmid DNA from approximately (˜1800) randomly selected clones was extracted by alkaline lysis (Feliciello and Chinali 1993). The DNA sequence of the selected clones was determined by single pass sequencing of the 5′ end using M13 reverse primer and the BigDye Terminator method (ABI Prism 310 DNA sequencer, Applied Biosystems) to generate expressed sequenced tags (ESTs). 10-12 ESTs were randomly selected and analysed for expression (Northern) under salinity stress conditions (0.5M NaCl) in Avicennia marina. One of these genes was found to be up-regulated in both leaves and roots of Avicennia marina under salinity stress. The clone was designated as Am244 and was used for further analysis. As a result of the sequence determination of the full length Am244 gene (SEQ ID NO: 1) it was found that cDNA was 600 bp in length.

### Example 3

### Expression of Am244 Gene in Response to Diverse Abiotic Stresses

NaCl Stress

One-month-old A. marina seedlings were conditioned for 72 hours in 0.5×MS nutrient solution with the roots dipping in the solution. Subsequently, plants were stressed with 0.5×MS containing 0.5M NaCl and leaf and root tissue frozen at 6, 12, 24 and 48 hours NaCl treatment and 12 and 24 hours after salt withdrawal. Leaf tissue was harvested and total RNA was isolated according to the method given by Chomczynski and Sacchi, 1987. The total RNA was then used for northern analysis. Total RNA was isolated as mentioned before. Equal amounts of total RNA (30 μg) were electrophoresed on a 1.5% MOPS-formaldehyde gel, transferred to nylon membrane (Hybond-N, Amersham) and fixed by UV cross linking according to the manufacturers instructions. PCR amplified product Am244 was labeled by the random primer method (Rediprime, Amersham) using α³²P-dCTP and used as probe. Radio-labeled probe were denatured and hybridized to the membrane at 65° C. in an aqueous buffer (5×SSC, 5% dextran sulphate, 0.05M Na-phosphate pH 7.2, 5×Denhardt's solution, 0.0025M EDTA, 0.4% SDS and 100 μg/ml salmon sperm DNA) for 12-16 h at 65° C. and washed for 15 min each with 2×SSC, 0.1% SDS and 1×SSC, 0.1% SDS. Hybridization signals were observed on Kodak X ray films after 2-3 days of exposure.

In A. marina roots, Am244 was rapidly upregulated, peaking at 6 hours of salt stress and being sustained thereafter up to 48 hours; upon withdrawal of the salt stress down regulation was also rapid. In the leaf however, the induction was more gradual and reached a peak at 48 hours of NaCl treatment; salt stress withdrawal did not bring about such a dramatic drop in the transcript level as was seen in the root. FIG. 1a shows up regulation of Am244 in response to salt stress in roots and leaves of A. marina. Am244 was then fully sequenced from both ends with universal M13F and M13R primers using the same BigDye terminator method mentioned above. The compiled Am244 sequence was then compared with those in the public database using BLASTX.

In one another experiment two month old A. marina seedlings were acclimatized in half strength MS salts for 2-3 days. Subsequently the seedlings were shifted to half strength MS salts supplemented with 500 mM NaCl. Leaf and root tissues were harvested prior to giving the stress (0 time point) and subsequently at intervals of 10′, 20′, 30′, 60′, two four and six hours after the application of NaCl stress. This stress application, unlike the previous one, was for a shorter time period. Total RNA was isolated as described previously. Northern blotting has been mentioned above.

In the leaf tissue of A. marine seedlings, there was a marginal induction of Am244 transcript seen at 20′ of NaCl application which increased at 30′ and was sustained at 60′. At 2 hours of NaCl application, the up-regulation of the Am244 transcript was four fold which was sustained up to 6 hours of stress examined. In the root tissue, the basal levels of expression of the Am244 transcript were higher than in leaf tissue. Upon application of stress, a two fold induction of the transcript was seen at one hour of stress that was sustained up to six hours (see FIG. 1b).

Abscisic Acid stress

For studying the effect of abscisic acid (ABA) treatment, one-month-old A. marina seedlings were acclimatized for 72 hours in 0.5×MS nutrient solution with the roots dipping in the solution. Subsequently, plants were treated with 0.5×MS containing 100 μM ABA and leaf and root tissue frozen at 6, 12, 24, 48 hours NaCl treatment and 12 and 24 hours after salt withdrawal. This tissue was similarly used for RNA isolation and subsequent Northern analysis.

As with salt stress, ABA treatment induced the Am244 gradually in the leaf tissue upon with maximum levels of transcript being observed at 48 hours of treatment. The expression level of the transcript dropped gradually with ABA withdrawal. In the roots, ABA treatment brought about negligible changes in Am244 expression. FIG. 1a shows expression profile of Am244 in response to ABA application in roots and leaves of A. marina

### KCl Stress

Two month old A. marina seedlings were acclimatized in half strength MS salts for 2-3 days. Subsequently the seedlings were shifted to half strength MS salts supplemented with 500 mM KCl. Leaf and root tissues were harvested prior to giving the stress (0 time point) and subsequently at intervals of 6, 10 and 24 hours after KCl treatment. Total RNA was isolated as described previously. Northern blotting has been mentioned before.

In the leaf tissue of A. marina seedlings, there was a two-fold induction of the Am244 transcript at six hours of KCl treatment that was sustained up to 24 hours. In the root tissue, there was a one-fold induction of the transcript at 10 hours of KCl treatment (FIG. 1b).

### Mannitol Stress

Two month old A. marina seedlings were acclimatized in half strength MS salts for 2-3 days. Subsequently the seedlings were shifted to half strength MS salts supplemented with 800 mM mannitol. Leaf and root tissues were harvested prior to giving the stress (0 time point) and subsequently at intervals of 6, 12 and 24 hours after mannitol treatment. Thereafter the seedlings were removed from the mannitol-containing medium and transferred to half strength MS salts only. Leaves and roots were harvested at 12 and 24 hours of mannitol withdrawal. Total RNA was isolated as described previously. Northern blotting has been mentioned before.

In the leaf tissue of A. marina seedlings, there was a three-fold induction of the Am244 transcript at 12 hours of mannitol treatment. Upon withdrawal of mannitol from the medium, transcript levels were seen to decline but not to basal levels (i.e. prior to mannitol application). In the root tissue, mannitol treatment led to a two-fold increase in the Am244 transcript that was maintained at 24 hours. Upon withdrawal of the mannitol from the medium, the transcript levels dropped below basal levels (FIG. 1b).

In all the experiments carried out for studying the regulation of Am244 gene in various abiotic stress conditions in A. marina the Am244 gene was found to be up-regulated.

### Example 4

### In Silico Sequence Analysis of Am244 cDNA

The Am244 cDNA sequence was subjected to BLASTX comparisons with the non-redundant protein database at the NCBI website (Altschul et al. 1990) to look for similarities for reported proteins. Protein translations from the nucleotide query sequence followed by amino acid sequence alignments for deduced protein against protein database is performed using BLASTX. The BLASTX algorithm uses the BLAST algorithm to compare the six-frame conceptual translation products of a nucleotide query sequence (both strands) against a protein sequence database. Default parameters of the program were used in all cases. A minimum P cutoff value of 10⁻³ (the probability that alignment would be generated randomly is 1<1000) was used to determine homology of Am244 to known proteins. The full length Am244 cDNA (SEQ ID NO: 1) sequence was analyzed for the presence of complete ORF using the ORF Finder program at the NCBI website. AN ORF of 57 amino acids (SEQ ID NO: 3) was identified in the Am244 cDNA. The Am244 ORF sequence is shown in SEQ ID NO: 2. This translated Am244 ORF was subjected to PSORT analysis to determine the presence of sorting signals within it Nakai and Horton, 1999). Similarly, HMMTOP analysis was undertaken to identify the presence of putative transmembrane segments (Tusnády and Simon, 1998). A search through the protein databases showed that homologs of the Am244 gene could be found in different groups including plants, animal fungi and bacteria, suggesting a common ancestor and a high degree of conservation through evolution. Am244 exhibits extensive similarity to proteins from Arabidopsis (RCI2A and RCI2B), barley (BLt101), wheat grass Lophopyrum (ESI3) and to predicted proteins from bacteria (YqaE), fungi (Ric1), nematodes (T23F23) and yeast (PmP3/SnA1) and belongs to the uncharacterized upf0057 family of putative plasma membrane proteins. CLUSTALW was utilized for generating multiple sequence alignment of both the full length Am244 cDNA sequence as well as the translated Am244 ORF along with its orthologs retrieved from the database to identify conserved and divergent blocks (Higgins D et al., 1994). As a result of the sequence determination of the full length Am244 gene (SEQ ID NO: 1) it was found that cDNA was 600 bp in length encoding a protein consisting of 57 amino acids (SEQ ID NO: 3). Four GATTT repeats are present in the 3′ untranslated region of the Am244 cDNA. PSORT analysis of Am244 gene product indicated putative plasma membrane localization and an uncleavable N-terminus signal peptide. The presence of the two transmembrane domains was predicted by HMMTOP.

### Example 5

### Construction of Recombinant Plant Transformation Vectors

Construction pGFP-Ala-Am244-C1 and pGFP-Ala-Am244-U1

GFP along with a flexible (Alanine)₁₀ linker was fused to the N-terminus of the Am244 cDNA fragment as shown in SEQ ID No: 2 by Splice Overlap Extension (SOEing). The use of the flexible (Alanine)₁₀ linker was based on the design by Cutler et al., 2000. The primers used for SOEing are as follows: Ala-SOE FWD (SEQ ID NO: 4), Ala-SOE REV (SEQ ID NO: 5), GFP FWD (SEQ ID NO: 6), GFP REV (SEQ ID NO: 7) and Am244 FUSION REV (SEQ ID NO: 8). The nucleotide sequence of the primers are given below.

SEQ ID NO: 4

5′ GCTGCCGCAGCTGCAGCCGCTATGGCTGAAGGGACAGCAACT 3′

SEQ ID NO: 5

5′ AGCGGCTGCAGCTGCGGCAGCTGCGGCTGCTTTGTATAGTTCATCCA
TGCCATG 3′

SEQ ID NO: 6

5′ CTAGTCTAGAATGAGTAAAGGAGAAGAACTTTTCA 3′

SEQ ID NO: 7

5′ CCGCTCGAGTTATTTGTATAGTTCATCCATGCCATG 3′

SEQ ID NO: 8

5′ CGC GGA TCC TCA GTC CCT GGT GAT GGC C 3′

All primers were diluted to a 100 ng/μl stock. Am244 cDNA fragment as shown in SEQ ID NO: 1 was cloned in the PstI and KpnI sites of pBluescript SK II. The construct thus derived was called pBSSK II-Am244. The clone for GFP (mGFP6) was obtained as part of a plasmid pMDC83 (Curtis M et al., 2003). The GFP fragment was digested with SacI and KpnI and the 0.75 Kb fragment cloned in the same sites in pBluescript SK II. The construct thus derived was called as pBSSK II-GFP. The pBSSK II-Am244 and pBSSK II-GFP clones were diluted to 1 ng/μl and were used as templates for amplification prior to SOEing.

I) Ala-SOE FWD and Am244 FUSION REV were used to amplify the Am244 cDNA fragment as shown in SEQ ID NO: 1 with the following reaction conditions: Reaction volume 50 μl; 1 mM total dNTPs, Am244 in PBSSK II (template) 10 ng/μlt; Ala-SOE FWD 150 ng; Am244 FUSION REV 100 ng, 2.5 U Pfu Turbo™ (Stratagene) in 1×Pfu Turbo buffer. PCR parameters were as follows: 94° C. for 3′ (pre-amplification denaturation); 18 PCR cycles of 94° C. for 1′, 59° C. for 1′, 68° C. for 2′; Final extension at 68° C. for 7′.

II) Ala-SOE REV and GFP FWD were used to amplify GFP with the following reaction conditions: Reaction volume 50 μl; 1 mM total dNTPs, GFP in PBSSK II (template) 10 ng/μl; Ala-SOE REV 150 ng; GFP FWD 100 ng, 2.5 U Pfu in 1×Pfu buffer. PCR parameters: 94° C. for 3′ (pre-amplification denaturation); 18 PCR cycles of 94° C. for 1′, 59° C. for 1′, 68° C. for 2′; Final extension at 6° C. for 7′.

Amplification (I) gave a band size of about 170 bp while amplification (II) gave a band size of about 720 bp. The amplification products were gel eluted and quantified. (I/Am244) and (II/GFP) were mixed in a 4.2:1.0 molar ratio (10 ng: 2.5 ng), heated to 68° C. for 2′ and chilled. This annealed product was used as template to amplify the GFP-(Alanine)₁₀-Am244 fragment. The reaction conditions for this were as follows: Reaction volume 50 μl; 1 mM total dNTPs, amplification (I/Am244) and amplification (II/GFP) products in a molar ratio of 4.2:1.0 (2.5 ng: 10 ng; GFP FWD 125 ng; Am244 FUSION REV 100 ng, 2.5 U Pfu in 1×Pfu buffer. PCR parameters: 94° C. for 3′ (pre-amplification denaturation); 18 PCR cycles of 94° C. for 1′, 59° C. for 1′, 68° C. for 2′; Final extension at 68° C. for 7′. Finally, a fragment of about 900 bp was obtained wherein 3′ GFP-(Alanine)₁₀ was fused to 5′ end of Am244 cDNA fragment as shown in SEQ ID NO: 2.

The fused fragment was gel eluted and cloned in Sma I site of pBSSK II to give rise to the plasmid called pBSSK II-GFP-Ala₁₀-Am244 The sequence of the GFP-(Alanine)₁₀-Am244 ORF was verified by sequencing with conventional primers such as M13F and M13R primers and also the primers Am244 FUSION REV, GFP FWD and GFP REV. The reads were subsequently compiled and checked for an intact ORF using translation tools available at EXPASY (www.expasy.com; Bairoch A et al, 2003). Following this, the 921 bp band was excised from pBSSK II using the restriction enzymes Bam III and Xba I and cloned in the pCAMBIA 1301+35S also digested with Bam HI and Xba I. These restriction sites were introduced in the Am244 FUSION REV and GFP FWD primers, respectively. The pCAMBIA 1301+35S is a modified binary plasmid vector where the CaMV 35S promoter is cloned in the Hind III and Xba I sites (multiple cloning site) of the binary plasmid vector pCAMBIA 1301 (1-Hajdukiewicz et al., 1994). The orientation of the GFP-(Alanine)₁₀-Am244 ORF in the pCAMBIA 1301+35S is shown in FIG. 2 and the construct has been named pGFP-Ala-Am244-C1.

Another construct designated as pGFP-Ala-Am244-U1 comprises of pCAMBIA 1301+Ubiquitin and the GFP-(Alanine)₁₀-Am244 ORF sequence cloned downstream of the ubiquitin promoter.

Constriction of Plant Transformation Vectors pAm244-C1, pAm244-C2, pAm244-U1, and pAm244-U2

Different plasmid vectors were constructed by cloning Am244 cDNA fragment as shown in SEQ ID NO: 1 or 2 in a modified binary vectors pCAMBIA 1301+CaMV35S to form pAm244-C1 and pAm244-C2 respectively. Similarly recombinant vectors pAm244-U1 and pAm244-U2 were constructed by cloning Am244 cDNA fragment as set forth in SEQ ID NO: 1 or 2 in modified binary vector pCAMBIA 1301+ubiquitin downstream of Ubiquitin promoter. The recombinant vector thus constructed were mobilized in the Agrobacterium cells by the freeze thaw method (Holsters M D et al., 1978) and used for the plant transformation.

Tobacco Transformation with pGFP-Ala-Am244-C1

Agrobacterium-mediated transformation of tobacco (Nicotiana tabacum) cv. Wisconsin was carried out by the standard protocol (Horsch R B et al., 1985). Briefly, sterile tobacco leaf discs were cut and transferred to Murashige and Skoog (MS) medium containing 3% sucrose, 1 mg/L BAP, 1 mg/L NAA, 0.8% Bacto-Agar, pH 5.6 at 28° C. in 16 hours light and 8 hours darkness for 24 hours prior to transformation. 100 ml of an overnight grown culture of pGFP-Ala-Am244-C1 transformed Agrobacterium strain was resuspended in 0.5×MS liquid medium with 3% sucrose, pH5.6 (5 ml). The leaf discs were subsequently co-cultivated with the Agrobacterium (transformed with pGFP-Ala-Am244-C1) for 30 minutes. The discs were dried on sterile No. 1 Whatmann discs and transferred to MS medium containing 3% sucrose, 1 mg/L Benzylaminopurine (BAP), 1 mg/L Napthaleneaceticacid (NAA), 0.8% Bacto-Agar, and pH 5.6 at 28° C. in 16 hours light and 8 hours darkness for 48 hrs. The leaf discs were given several washes in half strength liquid MS medium with 1.5% sucrose, pH 5.6 containing 250 mg/mL cefotaxime. Excess moisture on the leaf discs was blotted on sterile Whatmann No. 1 filter paper. The discs were then placed on selection media, that is, MS medium containing 3% sucrose, 1 mg/L BAP, 1 mg/L NAA, 0.8% Bacto-Agar, pH 5.6 containing 250 mg/mL cefotaxime and 25 mg/L hygromycin at 28° C. in 16 hours light and 8 hours darkness. The leaf discs were transferred to fresh selection media every 14 days until multiple shoot regeneration was seen. Shoot regeneration was seen between 20-45 days after first placing on the selection media. Regenerated independent shoots were then transferred to rooting medium (MS medium containing 3% sucrose, 0.8% Bacto-Agar, pH 5.6 containing 250 mg/mL cefotaxime and 25 mg/L hygromycin at 28° C. in 16 hours light and 8 hours darkness). After establishment of roots in the medium, the plants were transferred to fresh rooting medium every one month, each time transferring a shoot cut from the previous plant. Transformation of plants was confirmed by β-glucouronidase (GUS) staining of stem, leaf and root sections of the plant. The protocol for GUS staining was according to Jefferson R A et al. 1987. GUS positive plants comprising the for the pGFP-Ala-Am244-C1 T-DNA were then screened for GFP fluorescence using the Nikon Epifluorescence microscope.

### Example 6

### Epitope Tagging of the Am244 cDNA

The Am244 cDNA as shown in SEQ ID No. 1 cloned in the HindIII and PstI sites of pBSSK II (pBSSK-Am244) was used as template for epitope tag insertion. Two partially overlapping and complementary primers were designed to introduce the c-myc tag sequence in the middle of the Am244 cDNA and at the N-terminus of the Am244 ORF without disturbing the Am244 5′ UTR. The two primers used are Am244-Mut-Fwd (SEQ ID NO: 9) and A1-1244-Mut-Rev (SEQ ID NO: 10). Nucleotide sequence of these primers is given below.

SEQ ID NO: 9

5′GAACAAAAGTTGATTTCTGAAGAAGATCTGATGGCTGAAGGGACAGCA
ACTTGTATCGATATTG3′

SEQ ID NO: 10

5′CAGATCTTCTTCAGAAATCAACTTTTGTTCCATTTTTGCCTTCCCTTG
TTTGATTTTACCAAGAC3′

Am244-Mut-Fwd (SEQ ID NO: 9) codes for part of the c-myc tag (marked in bold) and the beginning Of the Am244 ORF. Am244-Mut-Rev (SEQ ID NO: 10) codes for the entire c-myc tag (marked in bold) as well as part of the Am244 5′ UTR.

### PCR Conditions

The insertion of the c-myc tag sequence within the Am244 cDNA was achieved according to Wang and Malcolm. Briefly, two single primer PCR reactions are carried out generating ‘hybrid’ linearized plasmids (with one wild type and one newly generated mutagenised strand). These linearized hybrid plasmids are then mixed and used as templates in the subsequent PCR steps to obtain hemi-methylated DNA as well as newly synthesized mutagenized non-methylated DNA. The parent and hemi-methylated DNA are removed by DpnI digestion while the newly synthesized mutagenized non-methylated DNA remains undigested. The reaction conditions employed were as follows: Two separate single primer reactions (25 μl each) were set up, one with the Am244-Mut-Fwd primer and the other with the Am244-Mut-Rev primer. The following components were mixed together template DNA (pBSSK-Am244)-50 ng; dNTPs-250 μM; 10×PCR buffer-2.5 μl, Am244-Mut-Fwd or Rev primers-12 pmoles and heated to 95° C. for 3′. 1.25 U of Pfu Polymerase was added and four PCR cycles were carried out under the following conditions: 95° C.-30s; 55° C.-1′; 68° C.-8′. The two single primer reactions were mixed and allowed to proceed for 16 more cycles under the following conditions: 95° C.-30s; 55° C.-1′; 68° C.-8′. 1 μl of DpnI was added to the reaction mix and held at 37° C. for 90′. 5 μl of the PCR reaction mix was used for transformation into ultracompetent XL-2 Blue cells. Seven colonies were obtained and were ‘size’ screened for the c-myc insertion using the universal M13R (SEQ ID NO: 11) and Am244 REV1 primer (SEQ ID NO: 12)

| | |
|---|---|
| 5′ AGCGGATAACATTTCACACA GG 3′ | SEQ ID NO: 11 |
| 5′ GATACAAGTTGCTGTCCCTTG 3′ | SEQ ID NO: 12 |

PCR reaction conditions: 95° C.-3′; 95° C.-30s, 57° C.-30s; 72° C.-30s (30 cycles); 72° C.-7′; 4° C.-∞. All seven colonies screened showed a larger fragment size (with a 33 bp insertion) as compared to the PCR-generated fragment obtained fromi pBSSK-Am244. Plasmids were prepared from all the seven colonies and sequenced with the M13R and M13F primers using terminator method. The 33 nucleotide insertion was found at the N-terminus of the Am244 ORF. This plasmid is referred to as pBSSK-mycAm244. pBSSK-mycAm244 was digested with BamHI and the excised myc-tagged Am244 cDNA cloned in the modified binary plasmid vector pCAMBIA1301+35S (where the CaMV 35S promoter was cloned in the Hind III and Xba I sites in the multiple cloning sites of the binary plasmid vector pCAMBIA 1301). The orientation of the myc-tagged Am244 cDNA in the plasmid vector pCAMBIA1301+35S is shown in FIG. 3 and the construct has been named pMyc-Am244-C1.

Similarly other recombinant transformation vector comprising c-myc tag sequence in the middle of the Am244 cDNA (SEQ ID NO: 1) operably linked to ubiquitin promoter. The recombinant vector thus constructed was designated as pMyc-Am244-U1.

Another recombinant vector comprising c-myc tag sequence at the N-terminus of the Am244 (SEQ ID NO: 2) operably linked to CaMV 35S promoter. The recombinant vector thus constructed was designated as pMyc-Am244-C2.

Another recombinant vector comprising c-myc tag sequence at the N-terminus of the Am244 (SEQ ID NO: 2) operably linked to ubiquitin promoter. The recombinant vector thus constructed was designated as pMyc-Am244-U2.

### Example 7

### Tobacco Transformation with pMyc-Am244-C1

Various recombinant plant transformation vectors describe as above were mobilizes into Agrobacterium strain LBA 4404 and EHA 105 and used for tobacco and rice transformation. The pMyc-Am244-C1 construct was mobilized into Agrobacterium tumefaciens strain LBA4404 by the freeze-thaw method.

Agrobacterium-mediated transformation of tobacco (Nicotiana tabacum) cv. Petit Havana was carried out by the standard protocol. Briefly, sterile tobacco leaf discs were cut and transferred to Murashige and Skoog (MS) medium containing 3% sucrose, 1 mg/L BAP, 1 mg/L NAA, 0.8% Bacto-Agar, pH 5.6 at 28° C. in 16 hours light and 8 hours darkness for 24 hours prior to transformation. 100 ml of an overnight grown culture of pGFP-Ala-Am244 transformed Agrobacterium strain was resuspended in 0.5×MS liquid medium with 3% sucrose, pH 5.6 (5 ml). The leaf discs were subsequently co-cultivated with the resuspended pMyc-Am244 transformed Agrobacterium for 30 minutes. The discs were dried on sterile No. 1 Whatmann discs and transferred to MS medium containing 3% sucrose, 1 mg/L BAP, 1 mg/L NAA, 0.8% Bacto-Agar, pH 5.6 at 28° C. in 16 hours light and 8 hours darkness for 48 hrs. The leaf discs were given several washes in liquid MS medium with 3% sucrose, pH 5.6 containing 250 mg/mL cefotaxime. Excess moisture on the leaf discs was blotted on sterile Whatmann No. 1 filter paper. The discs were then placed on selection media, that is, MS medium containing 3% sucrose, 1 mg/L BAP, 1 mg/L NAA, 0.8% Bacto-Agar, pH 5.6 containing 250 mg/mL cefotaxime and 25 mg/L hygromycin at 28° C. in 16 hours light and 8 hours darkness. The leaf discs were transferred to fresh selection media every 14 days until multiple shoot regeneration was seen. Shoot regeneration was seen between 20-35 days after first placing on the selection media. Regenerated independent shoots were then transferred to rooting medium (MS medium containing 3% sucrose, 0.8% Bacto-Agar, pH 5.6 containing 250 mg/mL cefotaxime and 25 mg/L hygromycin at 28° C. in 16 hours light and 8 hours darkness). After establishment of roots in the medium the plants transferred to fresh rooting medium every 14 days, each time transferring a shoot cut from the previous plant. Transformation of plants was confirmed by β-glucouronidase (GUS) staining of stem, leaf and root sections of the plant. The protocol for GUS staining was according to Jefferson R A et al, 1987. Twenty GUS positive lines were obtained from 23 independently transformed regenerants obtained. Leaves were harvested from these plants for genomic DNA isolation.

### Isolation of Genomic DNA

Genomic DNA from the transformed tobacco plant was isolated using the protocol of Richards EJ (1987). 2-3 grams of tobacco leaves (harvested from sixteen Myc-Am244 transformed lines as well as control untransformed tobacco cv. Petit Havana) were ground to a fine powder using liquid nitrogen in a mortar and pestle. The ground tissue was suspended in 8-10 ml of CTAB buffer (2% cetyltrimethylammoium bromide, 100 mM Tris-Cl pH 8.0, 20 mM EDTA, 1.4M NaCl and 0.2% β-mercaptoethanol) and incubated in a water bath at 65° C. for 30 minutes in centrifuge tubes. An equal volume of choloroform: isoamyl alcohol (24:1) was added and after inversion the tubes centrifuged at 10,000 rpm for 15° C. to allow for phase separation. The upper aqueous phase was transferred to a new tube and 0.6 volumes of isopropanol were added. The samples were left overnight at −20° C. for DNA precipitation. The tubes were then centrifuged at 12,000 rpm to pellet the DNA and the supernatant decanted. The pellet was allowed to air dry and resuspended in TE buffer (0.5 ml). The resuspended sample was treated with RNAse A (10 μg/ml) and incubated at 37° C. for two hours. An equal of phenol:choloroform (1:1) was added, mixed by inversion and centrifuged at 10,000 rpm for 10′ to separate the phases. The upper aqueous phase was transferred to a new Eppendorf and an equal volume of chloroform added, mixed by inversion and centrifuged at 10,000 rpm for 10′ to separate the phases. The upper aqueous phase was transferred to a new Eppendorf and 0.1 volume of 3M sodium acetate pH 5.2 was added followed by 2.5 volumes of absolute ethanol and kept for precipitation overnight at −20° C. The samples were then centrifuged at 12,000 rpm to pellet the genomic DNA and the supernatant discarded. The pellet was rinsed with 70% ethanol and centrifuged at 12,000 rpm, the wash discarded and allowed to air dry. The genomic DNA pellet was resuspended in TE.

PCR analysis was carried out with gene specific primers for confirming the presence of Am244 DNA in transgenic plants.

### Southern Hybridization

The genomic DNA (40 μg) isolated from sixteen lines Myc-Am244 lines and control (untransformed tobacco cv. Petit Havana) was digested with HindIII overnight. The digested genomic DNA was loaded on a 0.8% agarose gel (1×TBE) and run at 35 V for 16 hours. The gel was photographed and transferred to nylon membrane (Hybond N+, Amersham) using the alkaline transfer method mentioned in Sambrook et al. Briefly, the gel was incubated in denaturation solution (0.4N NaOH, 1M NaCl) for 30′. The capillary transfer method was used to transfer the genomic DNA to nylon membrane (Hybond N+, Amersham). Following transfer for 14-16 hours, the nylon membrane was rinsed twice with neutralization buffer (0.5M Tris-Cl pH 8.0, 1M NaCl) for 30′. The DNA was then cross-linked to the nylon blot was using a UV-crosslinker (Hoefer, UVC500). Pre-hybridization of the Southern blots was carried out at 56° C. in the phosphate hybridization buffer (0.5M Na₂HPO₄, 7% SDS and 1 mM EDTA @ 150 μl of buffer/cm² of the membrane) for 2 hours in a hybridization chamber (Hybridization Incubator Combi-H, Finemould Precision Inc.). 100 ng of PCR amplified and gel-purified Am244 cDNA was labeled in the presence of 5 μl of α-³²P-dCTP (BRIT, 3500 Ci/mmol) using the RediPrime Kit (Amersham) as per the manufacturer's instructions. The α-³²P-dCTP was purified through a Sephadex G-50 column to remove un-incorporated nucleotides. The probe was denatured for 5-7′ in boiling water, chilled on ice and added to the blot with fresh phosphate hybridization buffer (@ 150 μl of buffer/cm² of the membrane) and incubated at 56° C. for 14-16 hours in the hybridization oven. The membrane was washed with 2×SSC, 0.1% SDS for 15′ and exposed to the PhosphorImager screen (Personal Molecular Imager, BioRad) for 14-16 hours. The exposed images were scanned and analysed using the Quantity One software (BioRad).

The pMyc-Am244-C1 T-DNA has a single HindIII site flanking the 35S CaMV promoter driving the expression of myc-tagged Am244 cDNA. Digestion of the genomic DNA obtained from GUS positive Myc-Am244 transformed lines with HindIII would thus help in the identification of single copy insertions of the pMyc-Am244 T-DNA in the tobacco plants examined. Southern analysis of Myc-Am244 transformed lines revealed that there were single copy insertions of the Myc-Am244 cDNA in Lines 1, 6, 7, 8, 11 and 17.

### Example 8

### Transformation of Rice using Agrobacterium Mediated Method

Rice calli were transformed with the recombinant vectors disclosed in the invention by Agrobacterium-transformation methods as described in Example 5 and Example 7. Other standard protocols which are well known to the person skilled in art can also be used.

Rice calli was generated from mature seed scutella of pusa basmati 1 on a callus induction medium (MS inorganic+MS vitamins+2,4D (2 mg/ml)). Three week old calli were then infected with the Agrobacterium tumefaciens LBA4404 carrying any one of the recombinant vectors as described above such as pGFP-Ala-Am244-U1, pAm244-U1, pAm244-U2, pMyc-Am244-U1 or pMyc-Am244-U2. The infected calli were washed for a period of thirty minutes and then dried on a sterile filter paper. The dried calli were then transferred to a selection medium (callus induction medium containing Hygromycin (50 μg/ml)) for a period of 6 weeks. The calli were sub-cultured every fifteen days. The selected calli were then transferred to the regeneration medium (MS inorganic, MS vitamins, benzyl aminopurine (BAP (1.5 mg/L)), kinetin (0.5 mg/L) and NAA (0.5 mg/L)). The regenerants were transferred to the rooting medium (MS inorganic and MS vitamins without hormones). The platelets were subsequently transferred to the hardening medium for a span of two weeks and finally transferred to the soil in pots to raise the next generation of seeds. The tillers of the rice plants were bagged before the onset of flowering in order to promote self-pollination. The seeds from the selfed plants were collected and again sown for the next generation. The second generation rice paints were analyzed for the presence of the Am244 DNA as shown in SEQ ID NO: 1 or 2. The transgenic rice plants were screened for the localization of the polypeptide as shown in SEQ ID NO: 3.

These plants were tested for the presence of the Am244 DNA and also for copy number of the inserted gene. Further the plants were screened for tolerance to abiotic stress. The details are given in Example 10.

### Example 9

### Microscopy and Imaging

Leaf peels mounted in water were prepared from GUS positive tobacco and rice plants transformed with pGFP-Ala-Am244-C1 or pGFP-Ala-Am244-U1 respectively and examined under the Nikon Optiphot-2 phase contrast microscope fitted with an Episcopic fluorescence attachment (100 W Hg lamp). Fluorescence imaging of guard cells was carried out using the Nikon B2A filter set (excitation 450-490, Dichroic mirror 510, Barrier filter 520) and the Fluor 40× dry objective. Photographs were taken with a 35 mm FX-35DX camera using the Microflex HfX-DX attachment for automatic exposure adjustments on a Kodak ASA 400 film.

Guard cell imaging of pGFP-Ala-Am244-C1 and pGFP-Ala-Am244-C1 transformed tobacco and rice plants respectively showed localization of the green fluorescence at the periphery of the guard cells and close to the cell wall, suggesting plasma membrane localization of Am244. Chlorophyll auto-fluorescence was used to locate the chloroplasts under the same imaging conditions. Bright field images and fluorescence images of the tobacco guard cell are enclosed in FIG. 4.

### Example 10

### Expression Analysis of Transgenic Plant

### Whole-Plant Salt Stress Treatments:

The salt tolerance conferred by over-expressing Am244 gene in tobacco and rice transgenics was analyzed by performing whole plant salt stress treatments. Phenotypic growth retardation study was also performed between control and transgenic plants. Three control and transgenic plants were grown initially in ½ MS for 1 week. Later, they were transferred to ½ MS medium supplemented with 150 mM and 200 mM NaCl. It was observed that in 150 mM NaCl, transgenic plants showed better rooting when compared to control plants. At 200 mM NaCl, both control and transgenic plants did not root. It was also found that the transgenic plants suffered less damage in 150 mM and 200 mM NaCl stress. Phenotypic growth retardation was not evident in control and in both tobacco and rice transgenic plants grown in pots and irrigated with 150 mM NaCl solution for ½ week.

Similarly experiments were conducted for analyzing the transgenic plants for other stresses.

ABA stress: A stress of 1 μM ABA was given to control and transgenic plants (rice and tobacco) for same time intervals. It was observed that in 1 μM ABA, transgenic plants showed better rooting when compared to control plants which confirmed overexpression of Am244 DNA in rice and tobacco.

KCl stress: Control and transgenic were supplemented with 500 mM KCl. It was observed that in 500 mM KCl, transgenic plants showed better rooting when compared to control plants which confirmed overexpression of Am244 DNA in rice and tobacco.

Mannitol stress: Control and transgenic were supplemented with 800 mM KCl. It was observed that in 800 mM Mannitol, transgenic plants showed better rooting when compared to control plants which confirmed overexpression of Am244 DNA in rice and tobacco.

### REFERENCES

1. Altschul S F, Gish W, Miller W, Myers E W, Lipman D J (1990) Basic local alignment search tool. J Mol Biol 215:403-410
2. Boon P I, Allaway W G (1982) Assessment of leaf-washing techniques for measuring salt secretion in Avicennia marina (Forsk.) Vierh. Aust J Plant Physiol 9:725-734
3. Capel J, Jarillo J A, Salinas J, Martinez-Zapater J M (1997) Two homologous low-temperature-inducible genes from Arabidopsis encode highly hydrophobic proteins. Plant Physiol. 115: 569-76
4. Chauhan S, Forsthoefel N, Ran Y, Quigley F, Nelson D E, Bohncrt H J (2000) Na+/myo-inositol symporters and Na⁺/H⁺-antiporter in Mesembryanthemum crystallinum. Plant J 24:511-522
5. Chomczynski P, Sacchi N (1987) Single-step method of RNA isolation by acid guanidinium thiocyanate-phenol-chloroform extraction. Anal Bioclhem 162:156-159
6. Curtis M D, Grossniklaus U (2003) A gateway cloning vector set for high-throughput functional analysis of genes in planta. Plant Physiol. 133: 462-9
7. Cushman J C, Bohnert H J (2000) Genomic approaches to plant stress tolerance. Curr Opin Plant Biol 3:117-124
8. Cutler S R, Ehrhardt D W, Griffitts J S, Somerville C R (2000) Random GFP::cDNA fusions enable visualization of subcellular structures in cells of Arabidopsis at a high frequency. Proc Naitl Acad Sci USA. 97: 3718-23
9. Felicicllo I, Chinali G (1993) A modified alkaline lysis method for the preparation of highly purified plasmid DNA from Escherichia coli. Anal Biochem 212:394-401
10. Goddard N J, Dunn M A, Zhang L, White A J, Jack P L, Hughes M A (1993) Molecular analysis and spatial expression pattern of a low-temperature-specific barley gene, bIt101. Plant Mol Biol. 23: 871-9
11. Gulick P J, Shen W, An II (1994) ESI3, a stress-induced gene from Lopliopyrum elongatum. Plant Physiol. 104:799-800
12. Hajdukiewicz P Svab Z, Maliga, P (1994) The small versatile pPZP family of Agrobacterium binary vectors for plant transformation. Plant Mol Biol 25:989-994.
13. Higgins D, Thompson J, Gibson T, Thompson J D, Higgins D G, Gibson T J (1994) CLUSTAL W: improving the sensitivity of progressive multiple sequence alignment through sequence weighting, position-specific gap penalties and weight matrix choice. Nucleic Acids Res. 22:4673-4680.
14. Holsters M D, de Waele A, Depicker E, Messens M, Van Montagu, J Schell (1978) Transfection and transformation of A. tumefaciens. Mol. Gen. Genet. 163:181-187
15. Horsch R B, Fry J E, Hoffmann N L, Eichholtz D, Rogers S G, Fraley R T (1985) A simple and general method for transferring genes into plants. Science 227:1229-1231.
16. Jefferson R A, Kavanagh T A, Bevan M W (1987) GUS fusions: Beta-glucuroniidase as a sensitive and versatile gene fusion marker in higher plants. EMBO J 6:3901-3907.
17. Munns R, Husain S, Rivelli A R, James R A, Condon A G, Lindsay M P, Lagudah E S, Schachtman D P, Hare R A (2002) Avenues for increasing salt tolerance of crops, and the role of physiologically based selection traits. Plant Soil 247:93-105
18. Nakai K, Horton P (1999) PSORT: a program for detecting sorting signals in proteins and predicting their subcellular localization. Trends Biochem Sci. 24: 34-6.
19. Parani M (1999) Isolation of salt tolerance genes from mangroves: characterization of betaine aldehyde dehydrogenase (BADH) gene from Avicennia marina and its expression in tobacco trausgenic system. PhD thesis, Anna University, India
20. Rao A N (1987) Mangrove ecosystems of Asia and the Pacific. In: Umali R M, Zamora P M, Gotera R R, Jara R S, Camacho A S (eds) Mangroves of Asia and the Pacific: status and management. UNDP-UNESCO Technical Report, pp 1-41
21. Richards E J (1987) Preparation of genomic DNA from plant tissue. In: Current Protocols in Molecular Biology, (eds. F. Ausubel et al) John Wiley and Sons, New York, pp. 2.3.1-2.3.3.
22. Sambiroolk, J, Fritsch E F, and Maniatis T Molecular Cloning, 2nd ed. Cold Spring Harbor Laboratory Press, Cold Spring Harbor, N.Y.
23. Shimony C, Fahn A, Reinhold L (1973) Ultrastructure and ion gradients in the salt glands of Avicennia marina (Forsk.) Vierh. New Phytol 72:27-36
24. Tusnády GE and Simon I (1998) Principles Governing Amino Acid Composition of Integral Membrane Proteins: Applications to Topology Prediction.” J. Mol. Biol. 283: 489-506.
25. Van West P, Kamoun S, van't Klooster J W, Govers F (1999) Ric1, a Phytophthora infestans gene with homology to stress-induced genes. Curr Genet. 36: 310-5
26. Wang W, Malcolm 13A. Two-Stage Polymerase Chain Reaction Protocol Allowing Introduction of Multiple Mutations, Deletions, and insertions using QuikChange™ Site-directed mutagenesis. In: In vitro Mutagenesis Protocols. 2^{nd} ed. Series: Methods in Molecular Biology v. 182. Edited by J. Braman, Humana Press Inc. Totowa N.J.
27. Yale J, Bohnert H J (2001) Transcript expression in Saccharomyces cerevisiae at high salinity. J Biol Clhern. 276: 15996-6007
28. Zhu J K (2002) Salt and drought stress signal transduction in plants. Annu Rev Plant Biol 53:247-273

We claim:

1. An isolated nucleic acid molecule for enhanced tolerance to abiotic stress in plant having a nucleotide sequence with at least 90% homology to the nucleotide sequence set forth in SEQ ID NO: 1 or SEQ ID NO: 2, wherein said sequence codes for a polypeptide having amino acid sequence as shown in SEQ ID NO: 3.

2. The isolated nucleic acid molecule of claim 1, wherein said nucleic acid molecule comprises a nucleotide sequence as set forth in SEQ ID NO: 1 and SEQ ID NO:2.

3. A polypeptide having an amino acid sequence as set forth in SEQ ID NO: 3, wherein said polypeptide is encoded by the nucleic acid as claimed in claim 1.

4. The isolated nucleic acid molecule for enhanced tolerance to abiotic stress in a plant as claimed in claim 1, wherein said abiotic stress is selected from a group consisting of drought stress, salt stress and dehydration stress.

5. An expression cassette for enhanced tolerance to abiotic stress in plant, wherein said expression cassette comprises the nucleic acid molecule as claimed in claim 1 or claim 2 operably linked to a plant expressible regulatory sequence.

6. The expression cassette of claim 5, wherein the regulatory sequence is selected form a group consisting of CaMV 35S, NOS, OCS, AdhI, AdhII and Ubi-1.

7. A DNA construct comprising the expression cassette of claim 5.

8. The DNA construct as claimed in claim 7, wherein said construct further comprises another expression cassette comprising a selectable marker gene operably linked to the regulatory sequence as claimed in claim 6.

9. The DNA construct as claimed in claim 8, wherein the selectable marker gene is selected from a group consisting of nptII, hptII, pat and bar.

10. The DNA construct as claimed in claim 7 or claim 8, further comprises another expression cassette comprising a scorable marker gene operably linked to the regulatory sequence as claimed in claim 6.

11. The DNA construct as claimed in claim 10, wherein the scorable marker gene is selected from a group consisting of GUS, GFP, LUC and CAT.

12. A recombinant vector comprising the DNA construct of any one of the claim 7-11.

13. The recombinant vector as claimed in claim 12, wherein said vector is a plant transformation vector.

14. A recombinant host cell comprising the recombinant vector of claim 12.

15. The recombinant host cell as claimed in claim 14, wherein the host cell is a prokaryotic or eukaryotic cell.

16. The recombinant host cell of claim 15, wherein the prokaryotic cell is either E. coli or Agrobacterium.

17. The recombinant host cell as claimed in claim 15, wherein the eukaryotic cell is a plant cell.

18. An abiotic stress tolerant transgenic plant or plant cell or plant tissue comprising the nucleic acid molecule of claim 1, wherein the expression of the said nucleic acid molecule results in the enhanced tolerance to abiotic stress in said plant, plant cell and plant tissue.

19. The progeny derived from the transgenic plant as claimed in claim 18.

20. Transgenic seeds produced from the transgenic plant as claimed in claim 18, wherein said seed comprises the nucleic acid molecule of claim 1.

21. A method of producing a abiotic stress tolerant transgenic plant, said method comprising introducing at least one nucleic acid molecule of claim 1 in plant genome by using transformation method, thereby producing abiotic stress tolerant transgenic plant.

22. The method of claim 21, wherein said transformation method is selected from a group consisting of Agrobacterium mediated transformation, particle bombardment, vacuum-infiltration, in planta transformation and chemical method.

23. The method of claim 22, wherein the Agrobacterium mediated transformation comprising:
a) obtaining suitable explants from a plant,
b) constructing the recombinant vector of claim 12,
c) mobilizing said vector in Agrobacterium cell to produce recombinant Agrobacterium cell,
d) co-cultivating said explants with said recombinant Agrobacterium cell to produce transformed plant cells,
e) culturing said transformed plant cells to produce abiotic stress-tolerant transgenic plant.

24. The method of claim 21 or 23, wherein said plant is a monocotyledonous or a dicotyledonous plant.

25. The method of claim 24, wherein the monocotyledonous plant is selected from a group consisting of rice, maize, wheat, barley and sorghum.

26. The method of claim 25, wherein said monocotyledonous plant is rice plant.

27. The method of claim 24, wherein the dicotyledonous plant is selected from a group consisting of tobacco, tomato, pea, soybean, Brassica, chickpea and pigeon pea.

28. The method of claim 23, wherein said explants are selected from a group consisting of cotyledons, hypocotyls, leaves, anthers, callus, cotyledonary nodes, stems and roots.


SEQUENCE LISTING (13 sequences)

SEQ ID NO: 1 - DNA, 600 bp, Artificial Sequence
  Description of Artificial Sequence Synthetic polynucleotide
ggtagagaaaagagagataatcttgattctgtttaatttattttgtcttggtaaaatcaaacaagggaaggcaaaaatggctgaagggacagcaacttgtatcgatattgttgtggcaattcttctgcctccacttggtgtcttcctcaagtatggctgcaagggtgaattctggatttgtctgctactgaccatccttggttatattcctggaattatctatgctgtttgggccatcaccagggactgatatgttttccttgagaaggtatcctattcgattgtcaggtgaagatgcaaagggccataaagattgcagctttctttagcacacagacagtgctgatttgctggtgttaggctgcaagtccctttctcttccacttttgcttgtacccccttttccaatgtctcagatcactacttgatttgatttgatttgattttatcttgtcttattttgtatttaatgtgtgattaaagtgatttatcttagagtgtaagtcttggtttggccccactgtatatgcccctttagtaatgtaagtatggtaaccgtgaggtactacttactctatttaaaaaaaaaaaaaaaaaaaa

SEQ ID NO: 2 - DNA, 174 bp, Artificial Sequence
  Description of Artificial Sequence Synthetic polynucleotide
atggctgaagggacagcaacttgtatcgatattgttgtggcaattcttctgcctccacttggtgtcttcctcaagtatggctgcaagggtgaattctggatttgtctgctactgaccatccttggttatattcctggaattatctatgctgtttgggccatcaccagggactga

SEQ ID NO: 3 - protein, 57 aa, Avicennia marina
MAEGTATCIDIVVAILLPPLGVFLKYGCKGEFWICLLLTILGYIPGIIYAVWAITRD

SEQ ID NO: 4 - DNA, 42 bp, Artificial Sequence
  Description of Artificial Sequence Synthetic primer
gctgccgcagctgcagccgctatggctgaagggacagcaact

SEQ ID NO: 5 - DNA, 54 bp, Artificial Sequence
  Description of Artificial Sequence Synthetic primer
agcggctgcagctgcggcagctgcggctgctttgtatagttcatccatgccatg

SEQ ID NO: 6 - DNA, 35 bp, Artificial Sequence
  Description of Artificial Sequence Synthetic primer
ctagtctagaatgagtaaaggagaagaacttttca

SEQ ID NO: 7 - DNA, 36 bp, Artificial Sequence
  Description of Artificial Sequence Synthetic primer
ccgctcgagttatttgtatagttcatccatgccatg

SEQ ID NO: 8 - DNA, 28 bp, Artificial Sequence
  Description of Artificial Sequence Synthetic primer
cgcggatcctcagtccctggtgatggcc

SEQ ID NO: 9 - DNA, 64 bp, Artificial Sequence
  Description of Artificial Sequence Synthetic primer
gaacaaaagttgatttctgaagaagatctgatggctgaagggacagcaacttgtatcgatattg

SEQ ID NO: 10 - DNA, 65 bp, Artificial Sequence
  Description of Artificial Sequence Synthetic primer
cagatcttcttcagaaatcaacttttgttccatttttgccttcccttgtttgattttaccaagac

SEQ ID NO: 11 - DNA, 22 bp, Artificial Sequence
  Description of Artificial Sequence Synthetic primer
agcggataacatttcacacagg

SEQ ID NO: 12 - DNA, 21 bp, Artificial Sequence
  Description of Artificial Sequence Synthetic primer
gatacaagttgctgtcccttg

SEQ ID NO: 13 - protein, 10 aa, Artificial Sequence
  Description of Artificial Sequence Synthetic peptide
AAAAAAAAAA